(19) United States
(12) Patent Application Publication
(10) Pub. No.: US 2005/0187425 A1
(43) Pub. Date: Aug. 25, 2005

(54) Left ventricular function assist system and method

(75) Inventors: Clifton A. Alferness, Redmond, WA (US); John M. Adams, Sammamish, WA (US)

Correspondence Address: GRAYBEAL JACKSON HALEY LLP, Suite. 350, 155-108th Avenue N.E., Bellevue, WA, 98004-5973, US

(73) Assignee: Scout Medical Technologies, LLC

(21) Appl. No.: 11/035,011

(22) Filed: Jan. 12, 2005

Related U.S. Application Data
(60) Provisional application No. 60/536,565, filed on Jan. 14, 2004.

(51) Int. Cl. A61N 1/362; A61M 37/00

(52) U.S. Cl. 600/16

(57) ABSTRACT

A pump assists left ventricular function of a heart. The pump is configured for implant in the body and has an input connectable to the left atrium and an output connectable to the aorta for pumping blood from the left atrium to the aorta. A power source for powering operation of the pump is connectable to the pump. The pump may be configured for implant within the heart, such as, for example, in the atrium, the super vena cava, or partly within each of the right atrium and the superior vena cava.

### RELATED APPLICATION DATA

The present patent application is a continuation-in-part of Provisional U.S. Patent Application Ser. No. 60/536,565, filed Jan. 14, 2004.

### FIELD OF THE INVENTION

The present invention generally relates to a system and method for assisting the hemodynamic function of a heart. The present invention is more particularly directed to such a system and method wherein a pump is implanted in the heart and connected between the left atrium of the heart and the aorta for pumping blood from the left atrium to the aorta.

### BACKGROUND

Heart failure is one of the leading causes of death in the United States. Many remedies exist for reducing the effects of the failing heart of patients burdened by this malady. One therapy for treating the failing heart is to augment its hemodynamic function or pumping action by combining artificial pumping in parallel with the natural pumping action of the heart. This is accomplished with the surgical implantation of an entire system, referred to as a left ventricular assist device (LVAD) or portions thereof within the patient's chest.

The surgical procedure is a major, invasive, open chest surgical procedure. It is painful and costly. It also carries with it a substantial risk of death and other serious, although not fatal, complications and outcomes. Patients often must be cared for in the hospital for extended periods of time in order for their condition to stabilize to a point to enable them to just tolerate the implantation procedure. Post-operative hospital stays are also common for recovery from such surgeries.

It would therefore be a major medical and economic benefit if hemodynamic function assistance could be provided to the heart without the need for such major and invasive surgical procedures. The present invention addresses this and other issues which shall become apparent by providing a system and method which provides hemodynamic function assistance for the heart without the need for major, open chest, implantation procedures.

### SUMMARY OF THE INVENTION

The invention provides a system that assists left ventricular function of a heart having a superior vena cava, a left atrium, a right atrium, and an aorta. The system comprises a pump having an input connectable to the left atrium and an output connectable to the aorta. The pump is configured for implant in the body and pumps blood from the left atrium to the aorta. The system further comprises a power source for powering operation of the pump.

The system may further include a first fluid connector fluidly coupling the input of the pump to the left atrium and a second fluid connector fluidly coupling the output of the pump to the aorta. The first fluid connector may connect the pump to the left atrium through the interatrial septum between the left atrium and the right atrium. The second fluid connector may connect the pump to the non-coronary cusp of the aorta.

The pump may be configured for implant within the heart as, for example, within the right atrium. Alternatively, the pump may be configured for implant within the superior vena cava. The pump may comprise an axial flow pump.

The power source may comprise a rechargeable power source. The rechargeable power source may be a battery. The power source may alternatively be an electromagnetic energy source. The electromagnetic energy source may comprise an RF transmitter external to the heart and an RF receiver coupled to the pump.

The invention further provides a system that assists left ventricular function of a heart having a superior vena cava, a left atrium, a right atrium, an interatrial septum, and an aorta. The system comprises a first fluid connector fluidly coupling the left atrium and right atrium, a second fluid connector fluidly coupling the right atrium and the aorta, and a pump, configured for implant within the heart and to be coupled between the first and second fluid connectors for pumping blood from the left atrium into the aorta.

The invention further provides a method that assists left ventricular function of a heart having a superior vena cava, a left atrium, a right atrium, and an aorta. The method comprises the steps of implanting a pump in the heart, the pump having an input and an output, fluidly coupling the input of the pump to the left atrium, fluidly coupling the output of the pump to the aorta, and applying power to the pump to cause the pump to pump blood from the left atrium to the aorta.

The step of fluidly coupling the input of the pump to the left atrium may include the step of connecting the output of the pump to the left atrium through the interatrial septum between the left atrium and the right atrium. The step of fluidly coupling the output of the pump to the aorta may include the step of connecting the output of the pump to the non-coronary cusp of the aorta.

The implanting step may include the step of implanting the pump within the right atrium. The implanting step may alternatively include the step of implanting the pump within the superior vena cava.

The step of applying power to the pump may include the step of connecting a rechargeable power source to the pump. The step of applying power to the pump may alternatively include the steps of generating electromagnetic energy, converting the electromagnetic energy to a form of energy useable by the pump, and applying the form of energy useable by the pump to the pump.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a simplified representation of a patient with an associated left ventricular assist system according to one embodiment of the present invention;

FIG. 2 is a superior view of a heart with portions cut away and having a left ventricular assist pump implanted therein according to an embodiment of the present invention;

FIG. 3 is a superior view of a heart similar to FIG. 2 with an alternative left ventricular assist pump implanted therein according to a further embodiment of the present invention; and

FIG. 4 is a simplified schematic diagram of a rechargeable power source according to an embodiment of the present invention.

### DETAILED DESCRIPTION OF THE INVENTION

Referring now to FIG. 1, it shows a patient 10 having a system 40 assisting in the left ventricular function of the patient's heart 20 according to an embodiment of the present invention. The system 40 generally includes a pump 42 implanted within the heart 20 and a power source 44 for powering the pump 42.

The pump 42 may be an axial flow pump communicating between the left atrium of the heart and the aorta of the heart for pumping blood from the left atrium to the aorta. The pump may be implanted within the right atrium of the heart or within the superior vena cava of the heart, for example.

A power source 44, according to this embodiment, includes a transmitter 46 that transmits electromagnetic energy, such as radio frequency (RF) energy and a receiver 48 that receives the transmitted energy. The transmitter includes a wand antenna 52, similar to that used with external programmers capable of communicating with an implantable cardiac device. The receiver 48 may similarly comprise a wand antenna implantable just under the skin and circuitry of the type known in the art capable of converting the received electromagnetic energy into a form of energy usable by the pump 42. An example of such circuitry will be described hereinafter with reference to FIG. 4. Leads 54 couple the transmitter 46 to the antenna 52 and leads 56 couple the receiver 48 to the pump 42.

As may be seen subsequently, the power source may be a rechargeable power source to permit the patient 10 limited or temporary freedom of movement from the transmitter 46. The rechargeable power source may include a rechargeable battery with the receiver including circuitry for recharging the battery from the electromagnetic energy received from a non-implanted transmitter. Continuous power and/or battery recharging accommodates the continuous power load presented by the pump 42.

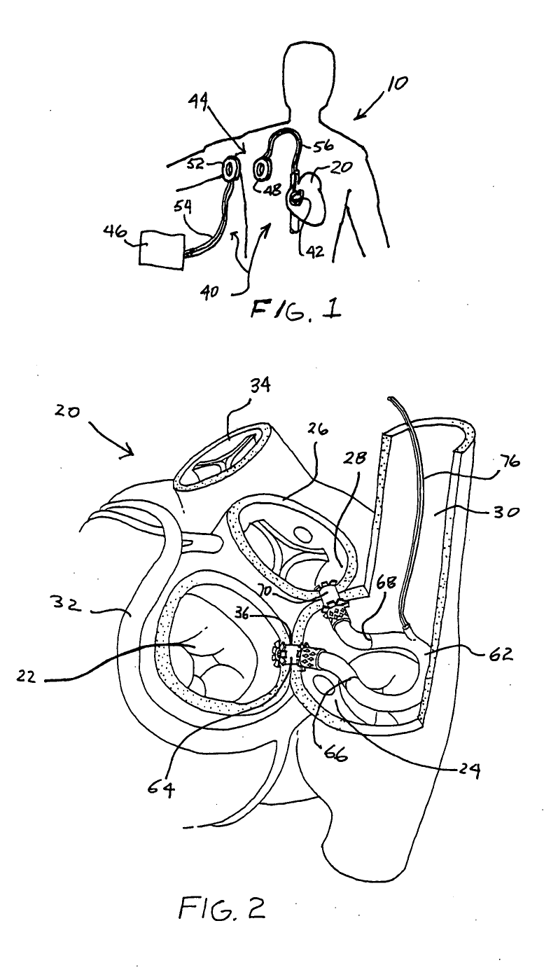

Referring now to FIG. 2, it shows a perspective view of the heart 20 having a pump 62 implanted in the right atrium according to one embodiment of the present invention. Portions of the heart 20 are cut away to reveal the components of the left ventricular assist system of FIG. 2.

The heart 20 is seen to include a left atrium 22, a right atrium 24, an aorta 26, a non-coronary cusp 28 of the aorta, and a superior vena cava 30. FIG. 2 also shows a coronary sinus 32 and a pulmonary valve 34.

To assist in the left ventricular function of the heart 20, the pump 62 is arranged to pump blood from the left atrium 22 to the aorta 26. To that end, the input 66 of the pump 62 is coupled to a first fluid connector 64 that connects the input 66 of the pump 62 to the left atrium 22. The connector 64 extends through the interatrial septum 36 between the left atrium 22 and the right atrium 24.

Similarly, the output 68 of the pump 62 is fluidly coupled to a second fluid connector 70. The second fluid connector 70 fluidly couples the pump output 68 to the non-coronary cusp 28 of the aorta 26. Leads 76 extend up through the superior vena cava to provide power from a power source to the pump 62. The power source may be the power source 44 previously described with respect to FIG. 1, or, for example, the power source of FIG. 4 to be described hereinafter.

The connectors may be structured and implanted in the heart as described, for example, in copending application Ser. No. 10/236,386, filed Sep. 6, 2002, for DEVICE AND METHOD PROVIDING ARTERIAL BLOOD FLOW FOR PERFUSION OF ISCHEMIC MYOCARDIUM and which has been published under Publication No. U.S. 2003/01811843 A1. The aforementioned published application is hereby incorporated herein by reference. The pump 62 may be an axial flow pump. Such pumps are well known and described, for example, in U.S. Pat. No. 6,533,716 B1 or U.S. Pat. No. 5,947,892, which patents are also incorporated by reference. The pumps preferably occupy less than twenty (20) cubic centimeters so as to fit within the right atrium. For example, the pump 62 may have a diameter of 1 cm and a length of 6 cm. The pump may be thus elongated and bendable as illustrated to further facilitate its implant down the superior vena cava within the heart and, in accordance with this embodiment, into the right atrium. With the pump implanted in the right atrium 24 and coupled between the left atrium 22 and the aorta 26, it may now be coupled to a power source by leads 76. The power applied to the pump will cause the pump to pump blood from the left atrium 22 to the aorta 26 to assist in the left ventricular function of the heart 20.

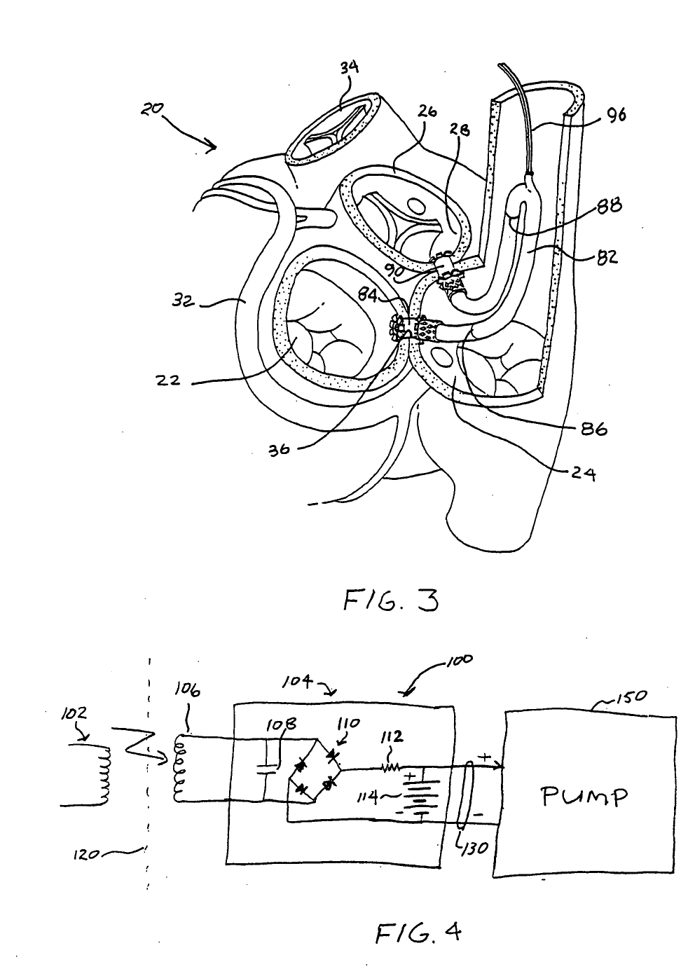

FIG. 3 shows another embodiment of the present invention to assist in the left ventricular function of the heart 20. Here, a pump 82 is implanted in the superior vena cava and arranged to pump blood from the left atrium 22 to the aorta 26. To that end, the pump 82 has an input 86 coupled to a first fluid connector 84 that connects the input 86 of the pump 82 to the left atrium 22. The first connector 84, as in the previous embodiment, extends through the interatrial septum 36 between the left atrium 22 and the right atrium 24.

Similarly, the output 88 of the pump 82 is fluidly coupled to a second fluid connector 90. The second fluid connector 90 fluidly couples the pump output 88 to the non-coronary cusp 28 of the aorta 26. Leads 96 extend up through the superior vena cava to provide power from a power source to the pump 62. The power source may again be the power source 44 previously described or, for example, the power source of FIG. 4 to be described hereinafter.

The connectors may be structured and implanted in the heart as described, for example, in the aforementioned and referenced published patent application Publication No. U.S. 2003/01811843 A1. The pump 82 may also be an axial flow pump as described, for example, in the previously referenced U.S. Pat. No. 6,533,716 B1 or U.S. Pat. No. 5,947,892. Again, the pumps preferably occupy less than 20 cubic centimeters so as to fit within the superior vena cava. Those skilled in the art will appreciate that the pump 82 may be alternatively implanted partly within the right atrium 24 and extend into the superior vena cava for also being partly within the superior vena cava 30 without departing from the present invention. With the pump implanted in the superior vena cava 30 and coupled between the left atrium 22 and the aorta 26, it may now be coupled to a power source by leads 96. The power will then cause the pump to pump blood from the left atrium to the aorta 26 to assist in the left ventricular function of the heart 20.

Referring now to FIG. 4, it shows a rechargeable power source 100 which may be employed according to an embodiment of the present invention to power any one of the left ventricular assist pumps disclosed herein and generally designated pump 150 in the figure. The power source 100 generally includes a transmitting coil antenna 102 and a receiver 104. As illustrated, the receiver 104 and pump 150 are implanted beneath the skin 120 of the patient. The pump 150 is, in accordance with this embodiment of the present invention, implanted within the heart as shown, for example, in FIG. 2 or 3, and the receiver is preferably implanted subcutaneously just beneath the skin 120 of the patient. The receiver is connected to the pump 150 by leads 130.

The transmitting antenna 120 may be employed for transmitting electromagnetic energy such as RF energy, for example, to the receiver 104. The receiver converts the RF energy to a form of energy which may be utilized by the pump 150 for sustaining its operation.

The receiver 104 includes a receiving coil antenna 106, a capacitor 108, and a bridge rectifier 110 of the type known in the art. The bridge rectifier 110, together with the capacitor 108, produce a pulsating DC current which is limited by a resistor 112. The pulsating DC limited current is then applied to a rechargeable battery 114. The battery 114 is coupled to the pump 150 by the leads 130.

By virtue of the rechargeable power source, the patient is provided with some limited freedom from the transmitting antenna 102. The pump 150 and/or the receiver 104 may have suitable control circuitry for controlling the application of the battery voltage to the pump and for alerting the patient that the need to recharge the battery is either eminent and/or presently required.

While particular embodiments of the present invention have been shown and described, modifications may be made. For example, the pump 42 need not necessarily be implanted within the heart. The pump 42 may still be implanted external to the heart but within the body and make connection to the left atrium and aorta as described herein. The conduits coupling the pump to the connectors within the heart may be transvenously brought outside of the heart to the pump. The pump may be more particularly placed, for example, in the chest, or elsewhere, where implantable cardiac devices are customarily implanted. Hence, it is therefore intended in the appended claims to cover all such changes and modifications which fall within the true spirit and scope of the invention.

1. A system that assists left ventricular function of a heart having a superior vena cava, a left atrium, a right atrium, and an aorta, the system comprising:
a pump configured for implant in the heart and having an input connectable to the left atrium and an output connectable to the aorta that pumps blood from the left atrium to the aorta; and
a power source for powering operation of the pump.

2. The system of claim 1, further comprising a first fluid connector fluidly coupling the input of the pump to the left atrium and a second fluid connector fluidly coupling the output of the pump to the aorta.

3. The system of claim 2, wherein the first fluid connector connects the pump to the left atrium through the interatrial septum between the left atrium and the right atrium.

4. The system of claim 2, wherein the second fluid connector connects the pump to the non-coronary cusp of the aorta.

5. The system of claim 1, wherein the pump is configured for implant within the right atrium.

6. The system of claim 1, wherein the pump is configured for implant within the superior vena cava.

7. The system of claim 1, wherein the power source comprises a rechargeable power source.

8. The device of claim 7, wherein the rechargeable power source is a battery.

9. The device of claim 1 wherein the power source is an electromagnetic energy source.

10. The device of claim 9, wherein the electromagnetic energy source comprises an RF transmitter external to the heart and an RF receiver coupled to the pump.

11. The system of claim 1, wherein the pump comprises an axial flow pump.

12. A system that assists left ventricular function of a heart having a superior vena cava, a left atrium, a right atrium, an interatrial septum, and an aorta, the system comprising:
a first fluid connector fluidly coupling the left atrium and right atrium;
a second fluid connector fluidly coupling the right atrium and the aorta; and
a pump, configured for implant within the heart and coupled between the first and second fluid connectors that pumps blood from the left atrium into the aorta.

13. The system of claim 12, wherein the first fluid connector connects the pump to the left atrium through the interatrial septum between the left atrium and the right atrium.

14. The system of claim 12, wherein the second fluid connector connects the pump to the non-coronary cusp of the aorta.

15. The system of claim 12, wherein the pump is configured for implant within the right atrium.

16. The system of claim 12, wherein the pump is configured for implant within the superior vena cava.

17. The system of claim 12, further comprising a power source that powers the pump.

18. The system of claim 17, wherein the power source comprises a rechargeable power source.

19. The system of claim 18, wherein the rechargeable power source is a battery.

20. The device of claim 17 wherein the power source is an electromagnetic energy source.

21. The device of claim 20, wherein the electromagnetic energy source comprises an RF transmitter external to the heart and an RF receiver coupled to the pump.

22. The system of claim 12, wherein the pump comprises an axial flow pump.

23. A method that assists left ventricular function of a heart having a superior vena cava, a left atrium, a right atrium, and an aorta, the method comprising:
implanting a pump in the heart, the pump having an input and an output;
fluidly coupling the input of the pump to the left atrium;
fluidly coupling the output of the pump to the aorta; and
applying power to the pump to cause the pump to pump blood from the left atrium to the aorta.

24. The method of claim 23, wherein the step of fluidly coupling the input of the pump to the left atrium includes the step of connecting the input of the pump to the left atrium through the interatrial septum between the left atrium and the right atrium.

25. The method of claim 23, wherein the step of fluidly coupling the output of the pump to the aorta includes the step of connecting the output of the pump to the non-coronary cusp of the aorta.

26. The method of claim 23, wherein the step of implanting the pump includes the step of implanting the pump within the right atrium.

27. The method of claim 23, wherein the step of implanting the pump includes step of implanting the pump within the superior vena cava.

28. The method of claim 23, wherein the step of applying power includes the step of connecting a rechargeable power source to the pump.

29. The method of claim 23 wherein the step of applying power includes the steps of generating electromagnetic energy, converting the electromagnetic energy to a form of energy useable by the pump, and applying the form of energy useable by the pump to the pump.

30. A system that assists left ventricular function of a heart having a superior vena cava, a left atrium, a right atrium, and an aorta, the system comprising:
a pump configured for implant in the body and having an input connectable to the left atrium and an output connectable to the aorta that pumps blood from the left atrium to the aorta; and
a power source for powering operation of the pump.

31. The system of claim 30, further comprising a first fluid connector fluidly coupling the input of the pump to the left atrium and a second fluid connector fluidly coupling the output of the pump to the aorta.

32. The system of claim 31, wherein the first fluid connector connects the pump to the left atrium through the interatrial septum between the left atrium and the right atrium.

33. The system of claim 31, wherein the second fluid connector connects the pump to the non-coronary cusp of the aorta.
